(19) United States
(12) Patent Application Publication
(10) Pub. No.: US 2013/0165946 A1
(43) Pub. Date: Jun. 27, 2013

(54) APPARATUS FOR A ROBOTIC CONTROL AND GUIDANCE SYSTEM

(71) Applicants: Kulbir S. Sandhu, Fremont, CA (US); Venkata Adusumilli, Santa Clara, CA (US); Jimmy Q. Duong, Millbrae, CA (US); Otto K. Chiu, Fremont, CA (US); Huaicai Mo, Milpitas, CA (US)

(72) Inventors: Kulbir S. Sandhu, Fremont, CA (US); Venkata Adusumilli, Santa Clara, CA (US); Jimmy Q. Duong, Millbrae, CA (US); Otto K. Chiu, Fremont, CA (US); Huaicai Mo, Milpitas, CA (US)

(21) Appl. No.: 13/335,184

(22) Filed: Dec. 22, 2011

(51) Int. Cl. A61B 19/00

(52) U.S. Cl. 606/130

(57) ABSTRACT

An apparatus for a robotically controlled medical device guidance system comprises a manipulator assembly and a medical device cartridge. The manipulator assembly includes a housing, an electrical port having a first end configured for mating with a complementary electrical connector of a cable, and a manipulation base disposed within the housing and including an electrical interface. The electrical interface is electrically connected to a second end of the electrical port. The cartridge is configured to be removably attached to the manipulation base. The cartridge includes an electrical interface configured to electrically contact the electrical interface of the manipulation base when the cartridge is attached to the base, and a medical device associated therewith. The medical device includes a sensor and a corresponding lead wire. The lead wire is electrically connected to and between the sensor and the electrical interface of the cartridge.

### BACKGROUND OF THE INVENTION

a. Field of the Invention

The present disclosure relates generally to a robotic control and guidance system (RCGS) for one or more medical devices. More particularly, the present disclosure relates to apparatus, such as, for example, a manipulator assembly and a medical device cartridge, for a RCGS.

b. Background Art

Electrophysiology (EP) catheters are used in a variety of diagnostic and/or therapeutic medical procedures to correct conditions such as atrial arrhythmia, including for example, ectopic atrial tachycardia, atrial fibrillation, and atrial flutter. Arrhythmia can create a variety of dangerous conditions including irregular heart rates, loss of synchronous atrioventricular contractions and stasis of blood flow which can lead to a variety of ailments.

In a typical EP procedure, a physician manipulates a catheter through a patient's vasculature to, for example, a patient's heart. The catheter typically carries one or more electrodes that may be used for mapping, ablation, diagnosis, and the like. Once at the target tissue site, the physician commences diagnostic and/or therapeutic procedures, for example, ablative procedures such as radio frequency (RF), microwave, cryogenic, laser, chemical, acoustic/ultrasound or high-intensity focused ultrasound (HIFU) ablation, to name a few different sources of ablation energy. The resulting lesion, if properly located and sufficiently contiguous with other lesions, disrupts undesirable electrical pathways and thereby limits or prevents stray electrical signals that can lead to arrhythmias. Such procedures require precise control of the catheter during navigation, and delivery of therapy, to the target tissue site, which can invariably be a function of a user's skill level.

Robotic control and guidance systems (RCGS) for one or more medical devices (or robotically controlled medical device guidance systems) are known to facilitate such precise control. In general, these types of systems carry out (as a mechanical surrogate) input commands of a clinician or other end-user to deploy, navigate, and manipulate one or more medical devices, such as, for example, a catheter and/or an introducer or sheath for a catheter, or some other elongate medical instrument. One exemplary robotic catheter system is described and depicted in U.S. Patent Publication No. 2009/0247993 entitled “Robotic Catheter System,” the entire disclosure of which is incorporated herein by reference.

A conventional RCGS typically includes, among other components, a manipulator assembly and one or more medical device cartridges. Each medical device cartridge has a medical device (e.g., a catheter or sheath) coupled therewith, and is configured to be mounted on, or attached to, a corresponding manipulation base of the manipulator assembly. When a medical device cartridge is attached to the manipulation base, the manipulator assembly is configured to maneuver the medical device associated with the medical device cartridge by, for example, translating the device cartridge back and forth in a linear fashion.

A medical device associated with a medical device cartridge often will include one or more sensors disposed at or near the distal end of the device. The sensors may be used for a variety of diagnostic and therapeutic purposes including, for example, EP studies, catheter identification and location, pacing, cardiac mapping, ablation, and the like. Each sensor includes at least one lead wire. The lead wire(s) are configured to electrically connect the sensor with an electrical interface associated with the cartridge to allow data or signals acquired or produced by the sensors to be communicated to other components of the RCGS. More particularly, in conventional systems, the electrical interface associated with the cartridge takes the form of an electrical cable extending from the housing of the cartridge. The cable includes an electrical connector disposed at the end of the cable opposite the end that interfaces with the cartridge that is configured to be mated with a complementary connector of another electrical cable that is, in turn, electrically connected to another components of the RCGS. Accordingly, the data or signals acquired or produced by the sensor is communicated through the lead wire(s) to the electrical interface disposed external to the cartridge, and to another component of the RCGS through a cable connecting the electrical interface with that particular RCGS component.

Conventional systems such as that briefly described above are not without their drawbacks, however. For instance, as described above, the medical device cartridge is configured to be moved by the manipulator assembly in order to maneuver the associated medical device. Because the cable comprising the electrical interface for electrically connecting the sensors on the medical device with one or more other components of the RCGS extends from the cartridge, it also moves along with the cartridge. Additionally, because the electrical interface comprises a cable extending from the cartridge, it is disposed external to the cartridge. As such, it is possible that either the interface cable of the cartridge or the cable connected to the interface cable can get tangled with other electrical cables or structure of, or in close proximity to, the RCGS, and as a result, the interface cable and the cable connected thereto can become disconnected. If this occurs, it could lead to, among other undesirable consequences, significant and substantial delays in the procedure being performed by the RCGS.

Additionally, in view of the location of the electrical interface of the cartridge, each time the cartridge has to be replaced the electrical interface must be manually disconnected prior to detaching the cartridge from the manipulator assembly, the cartridge is then detached from the manipulator assembly, the new cartridge is attached to the manipulator assembly, and then the electrical cable that was attached to the electrical interface of the original cartridge must be connected to the electrical interface of the new cartridge. This results in a cartridge-changing process that is undesirably complex.

Accordingly, the inventors herein have recognized a need for apparatus, such as, for example, a manipulator assembly and a medical device cartridge, for a RCGS that will minimize and/or eliminate one or more of the deficiencies in conventional systems.

### BRIEF SUMMARY OF THE INVENTION

The present invention is directed to apparatus for a robotic control and guidance system.

In accordance with one aspect of the invention and the present teachings, an apparatus for a robotically controlled medical device guidance system comprises a manipulator assembly having a housing, an electrical port, and a manipulation base disposed within the housing and including an electrical interface. The electrical port comprises a first end configured for mating with a complementary electrical connector of an electrical cable. In an exemplary embodiment, the electrical port is disposed in the housing. In another exemplary embodiment, the apparatus further comprises a support structure for supporting the manipulator assembly, and the port is disposed in the support structure. The electrical interface of the manipulation base is electrically connected to a second end of the electrical port. In an exemplary embodiment, the electrical interface comprises a contact pad a plurality of electrical contacts disposed thereon. In another exemplary embodiment, the electrical interface comprises at least one pin or at least one socket. The apparatus further comprises a medical device cartridge.

The medical device cartridge is configured to be removably attached to the manipulation base, and has an electrical interface configured to electrically contact the electrical interface of the manipulation base when the medical device cartridge is attached to the manipulation base. In an exemplary embodiment, the electrical interface comprises a contact pad having a plurality of electrical contacts that are complementary to the electrical contacts of the contact pad of the manipulation base electrical interface. In another exemplary embodiment, the electrical interface comprises at least one pin or at least one socket. The medical device cartridge further includes a medical device associated therewith that includes a sensor and a corresponding lead wire. In an exemplary embodiment, the medical device is one of a catheter and a sheath. In either instance, the lead wire is electrically connected to and between the sensor and the electrical interface of the medical device cartridge. In an exemplary embodiment, the medical device cartridge further comprises a memory, such as, for example, an EEPROM, containing identifying data relating to the medical device. In an exemplary embodiment, the memory is electrically connected to the electrical interface of the medical device cartridge. The manipulation base of the manipulator assembly may further include a sensor configured to detect whether the medical device cartridge is attached thereto.

In an exemplary embodiment, the medical device cartridge is attached to the manipulation base, and the electrical interface of the medical device cartridge is in electrical contact with the electrical interface of the manipulation base. As a result of this electrical connection between the electrical interfaces, the sensor of the medical device is electrically connected to the electrical port of the manipulator assembly.

In another exemplary embodiment, the electrical port of the manipulator assembly is a first electrical port, the manipulation base is a first manipulation base, and the medical device cartridge is a first medical device cartridge. In such an embodiment, the manipulator assembly further includes a second electrical port, a first end of which is configured for mating with a complementary electrical connector of an electrical cable. In an exemplary embodiment, the second port is disposed in the housing. In another exemplary embodiment, the apparatus further comprises a support structure for supporting the manipulator assembly, and the second port is disposed in the support structure. The manipulator assembly further includes a second manipulation base disposed within the housing thereof. The second manipulation base includes an electrical interface that is electrically connected to a second end of the second electrical port of the manipulator assembly. In this embodiment, the apparatus further comprises a second medical device cartridge configured to be removably coupled with the second manipulation base. The second medical device cartridge includes an electrical interface configured to electrically contact the electrical interface of the second manipulation base when the second medical device cartridge is attached to the second manipulation base. The second medical device cartridge further comprises a medical device associated therewith that includes a sensor and a corresponding lead wire, wherein the lead wire is electrically connected to and between the sensor and the electrical interface of the second medical device cartridge.

In an exemplary embodiment, the first medical device cartridge is attached to the first manipulation base, and the second medical device cartridge is attached to the second manipulation base. In such an embodiment, the electrical interface of the first medical device cartridge is in electrical contact with the electrical interface of the first manipulation base, and as a result, the sensor of the medical device of the first medical device cartridge is electrically connected to the second end of first electrical port. Similarly, the electrical interface of the second medical device cartridge is in electrical contact with the electrical interface of the second manipulation base, and as a result, the sensor of the medical device of the second medical device cartridge is electrically connected to the second end of the second electrical port.

In accordance with another aspect of the invention, an apparatus for a robotically controlled medical device guidance system comprising a housing, an electrical port disposed in the housing, and a manipulation base disposed within the housing is provided. The electrical port comprises a first end that is configured for mating with a complementary electrical connector of an electrical cable. The manipulation base is configured to have a medical device cartridge removably attached thereto, and includes an electrical interface configured to electrically contact a corresponding electrical interface of the medical device cartridge when the medical device cartridge is attached thereto. The electrical interface, which may comprise a contact pad having a plurality of electrical contacts, is electrically connected to a second end of the electrical port. In another exemplary embodiment, the electrical interface comprises a plurality of pins or a plurality of sockets. The manipulation base may further comprise a sensor configured to detect whether a medical device cartridge is attached thereto.

In another exemplary embodiment, the electrical port is a first electrical port and the manipulation base is a first manipulation base. In such an embodiment, the manipulator assembly further comprises a second electrical port disposed in the housing, a first end of which is configured for mating with a complementary electrical connector of an electrical cable. The manipulator assembly further still further comprises a second manipulation base disposed within the housing configured to have a medical device cartridge removably attached thereto, and including an electrical interface configured to electrically contact a corresponding electrical interface of the medical device cartridge when the medical device cartridge is attached to the second manipulation base. The electrical interface of the second manipulation base is electrically connected to a second end of the second electrical port.

In accordance with another aspect of the invention, a medical device cartridge for a robotically controlled medical device guidance system is provided. The medical device cartridge comprises a housing having a top side and a bottom side, wherein the bottom side is configured to permit the medical device cartridge to be removably attached to a manipulator assembly of the robotically controlled medical device guidance system. The medical device cartridge further includes an electrical interface, which may comprise a contact pad comprising a plurality of electrical contacts, disposed on the bottom side of the housing that is configured to electrically contact a corresponding electrical interface associated with the manipulator assembly when the medical device cartridge is attached thereto. The medical device cartridge further includes a medical device, which may comprise, for example, a catheter or a shaft. The medical device has a proximal end and a distal end, wherein the proximal end is retained within the housing of the medical device cartridge. Additionally, the medical device further includes a sensor and a corresponding lead wire, wherein the lead wire is electrically connected to and between the sensor and the electrical interface.

In an exemplary embodiment, the medical device cartridge further includes a memory, which may comprise an EEPROM, containing identifying data relating to the medical device. In an exemplary embodiment, the memory is electrically connected to the electrical interface of the medical device cartridge.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 is an isometric diagrammatic view of a robotic control and guidance system, and a robotically controlled medical device guidance system, in particular, illustrating an exemplary layout of various system components.

FIGS. 2a is a side view of an exemplary manipulator assembly of the system illustrated in FIG. 1 coupled to a robotic support structure, wherein the manipulator assembly has an electrical port in the housing thereof.

FIG. 2b is a side view of another exemplary manipulator assembly of the system illustrated in FIG. 1 coupled to a robotic support structure, wherein the robotic support structure includes an electrical port.

FIG. 3 is an isometric view of the exemplary manipulator assembly illustrated in FIG. 2a, with portions of the housing thereof removed to show the exemplary manipulator assembly in greater detail.

FIG. 4 is an isometric view showing the rear portion of the manipulator assembly shown in FIG. 3, and electrical ports in the housing of the manipulator assembly, in particular.

FIGS. 5a and 5b are elevation views of exemplary embodiments of the electrical ports of the manipulator assembly shown in FIG. 4.

FIG. 6 is an isometric view of an exemplary manipulation base of the manipulator assembly shown in FIG. 3.

FIG. 7 is a diagrammatic and schematic view of the electrical connections between an exemplary electrical interface of a manipulation base illustrated in FIG. 6 and an electrical port disposed in the housing of the manipulator assembly.

FIG. 8a is an isometric view of an exemplary catheter cartridge for use with the manipulator assembly shown in FIG. 3.

FIG. 8b is another isometric view of the catheter cartridge shown in FIG. 8a, showing the bottom side thereof in greater detail.

FIGS. 9a is a side view of another exemplary manipulator assembly of the system illustrated in FIG. 1 coupled to a robotic support structure, wherein the manipulator assembly has a cable extending therefrom.

FIG. 9b is a side view of yet another exemplary manipulator assembly of the system illustrated in FIG. 1 coupled to a robotic support structure, wherein the robotic support structure has a cable extending therefrom.

### DETAILED DESCRIPTION OF THE INVENTION

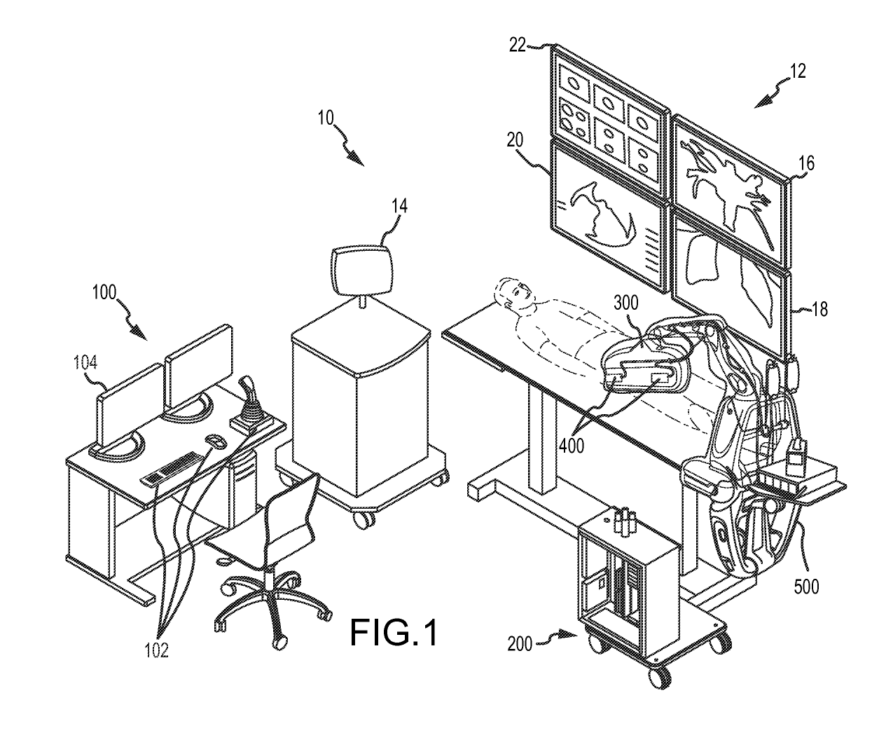

Referring now to the drawings wherein like reference numerals are used to identify identical components in the various views, FIG. 1 illustrates one exemplary embodiment of a robotic control and guidance system 10 (RCGS 10) for manipulating one or more medical devices. The RCGS 10 can be used, for example, to manipulate the location and orientation of catheters and sheaths in a heart chamber or in another body cavity or lumen. The RCGS 10 thus provides the user with a similar type of control provided by a conventional manually-operated system, but allows for repeatable, precise, and dynamic movements. For example, a user such as an electrophysiologist can identify locations (potentially forming a path) on a rendered computer model of the cardiac anatomy. The system can be configured to relate those digitally selected points to positions within a patient's actual/physical anatomy, and can thereafter command and control the movement of the sheath and/or catheter to the defined positions. Once at the specified target position, either the user or the system can perform the desired diagnostic or therapeutic function. The RCGS 10 enables full robotic navigation/guidance and control.

As shown in FIG. 1, the RCGS 10 can generally include one or more monitors or displays 12, a visualization, mapping, and/or navigation system 14, a human input device and control system (referred to as “input control system”) 100, an electronic control system 200, a manipulator assembly 300 for operating one or more device cartridges 400, and a manipulator support structure 500 for positioning the manipulator assembly 300 in proximity to a patient or a patient's bed.

The displays 12 are configured to visually present to a user information regarding patient anatomy, medical device location or the like, originating from a variety of different sources. The displays 12 can include (1) a monitor 16 (coupled to system 14—described more fully below) for displaying cardiac chamber geometries or models, displaying activation timing and voltage data to identify arrhythmias, and for facilitating guidance of catheter movement; (2) a fluoroscopy monitor 18 for displaying a real-time x-ray image or for assisting a physician with catheter movement; (3) an intra-cardiac echo (ICE) display 20 to provide further imaging; and (4) an EP recording system display 22.

The visualization, navigation, and/or mapping system 14 is configured to provide many advanced features, such as visualization, mapping, navigation support and positioning (i.e., determine a position and orientation (P&O) of a sensor-equipped medical device, for example, a P&O of a distal tip portion of a catheter). In an exemplary embodiment, the system 14 may comprise an impedance-based system, such as, for example, the EnSite NavX™ system commercially available from St. Jude Medical, Inc., and as generally shown by reference to U.S. Pat. No. 7,263,397 entitled “Method and Apparatus for Catheter Navigation and Location and Mapping in the Heart,” the entire disclosure of which is incorporated herein by reference. In other exemplary embodiments, however, the system 14 may comprise other types of systems, such as, for example and without limitation: a magnetic-field based system such as the Carto™ System available from Biosense Webster, and as generally shown with reference to one or more of U.S. Pat. No. 6,498,944 entitled “Intrabody Measurement,” 6,788,967 entitled “Medical Diagnosis, Treatment and Imaging Systems,” and U.S. Pat. No. 6,690,963 entitled “System and Method for Determining the Location and Orientation of an Invasive Medical Instrument,” the entire disclosures of which are incorporated herein by reference, or the gMPS system from MediGuide Ltd., and as generally shown with reference to one or more of U.S. Pat. Nos. 6,233,476 entitled “Medical Positioning System,” U.S. Pat. No. 7,197,354 entitled “System for Determining the Position and Orientation of a Catheter,” and U.S. Pat. No. 7,386,339 entitled “Medical Imaging and Navigation System,” the entire disclosures of which are incorporated herein by reference; and a combination impedance-based and magnetic field-based system such as the Carto 3™ System also available from Biosense Webster.

As briefly described above, in an exemplary embodiment, the system 14 involves providing one or more positioning sensors for producing signals indicative of medical device location (position and/or orientation) information. In an embodiment wherein the system 14 is an impedance-based system, the sensor(s) may comprise one or more electrodes. Alternatively, in an embodiment wherein the system 14 is a magnetic field-based system, the sensor(s) may comprise one or more magnetic sensors (e.g., coils) configured to detect one or more characteristics of a low-strength magnetic field.

The input control system 100 is configured to allow a user, such as an electrophysiologist, to interact with the RCGS 10, in order to control the movement and advancement/withdrawal of one or more medical devices, such as, for example, a catheter and/or a sheath (see, e.g., U.S. Patent Publication No. 2010/0256558 entitled “Robotic Catheter System,” and PCT/US2009/038597 entitled “Robotic Catheter System with Dynamic Response,” published as WO 2009/120982, the entire disclosures of which are incorporated herein by reference). Generally, several types of input devices and related controls can be employed, including, without limitation, instrumented traditional catheter/sheath handle controls, oversized catheter/sheath models, instrumented user-wearable gloves, touch screen display monitors, 2-D input devices, 3-D input devices, spatially detected styluses, and traditional joysticks. For a further description of exemplary input apparatus and related controls, see, for example, U.S. Patent Publication Nos. 2011/0015569 entitled “Robotic System Input Device” and 2009/0248042 entitled “Model Catheter Input Device,” the entire disclosures of which are incorporated herein by reference. The input devices can be configured to directly control the movement of the catheter and sheath, or can be configured, for example, to manipulate a target or cursor on an associated display.

The electronic control system 200 is configured to translate (i.e., interpret) inputs (e.g., motions) of the user at an input device of the input control system 100 (or from another source) into a resulting movement of one or more medical devices (e.g., a catheter and/or a sheath). In this regard, the system 200 includes a programmed electronic control unit (ECU) in communication with a memory or other computer readable media (memory) suitable for information storage. Relevant to the present disclosure, the electronic control system 200 is configured, among other things, to issue commands (i.e., actuation control signals) to the manipulator assembly 300 (i.e., to the actuation units—electric motors thereof, in particular) to move or bend the medical device(s) associated therewith to prescribed positions and/or in prescribed ways, all in accordance with the received user input and/or a predetermined operating strategy programmed into the system 200. In addition to the instant description, further details of a programmed electronic control system can be found in U.S. Patent Publication No. 2010/0256558, the entire disclosure of which was incorporated herein by reference above. It should be understood that although the visualization, navigation, and/or mapping system 14 and the electronic control system 200 are shown separately in FIG. 1, integration of one or more computing functions can result in a system including an ECU on which can be run both (i) various control and diagnostic logic pertaining to the RCGS 10 and (ii) the visualization, navigation, and/or mapping functionality of system 14.

The manipulator assembly 300, in response to commands issued by the electronic control system 200, is configured to maneuver the medical device(s) associated therewith (e.g., translation movement, such as advancement and withdrawal of the medical device(s)), as well as to effectuate distal end (tip) deflection and/or rotation or virtual rotation. In an embodiment, the manipulator assembly 300 can include actuation mechanisms/units (e.g., a plurality of electric motor and lead screw combinations, or other electric motor configurations) for linearly actuating one or more control members (e.g., steering wires) associated with the medical device(s) for achieving the above-described translation, deflection and/or rotation (or virtual rotation). In addition to the description set forth herein, further details of a manipulator assembly can be found in U.S. Patent Publication No. 2009/0247942 entitled “Robotic Catheter Manipulator Assembly,” the entire disclosure of which is incorporated herein by reference.

A device cartridge 400 is provided for each medical device controlled by the RCGS 10. For this exemplary description of an RCGS, and as will be described in greater detail below, one cartridge is associated with a catheter and a second cartridge is associated with an outer sheath. It will be appreciated, however, that in other exemplary embodiments, a single medical device, and therefore, a single cartridge, or more than two medical devices, and therefore, more than two cartridges, may be used. Accordingly, embodiments wherein more or less than two cartridges are employed remain within the spirit and scope of the present disclosure. In any event, the cartridges are coupled, generally speaking, to the RCGS 10 to allow for robotically-controlled movement. In addition to the description set forth herein, further details of a device cartridge can be found in U.S. Patent Publication Nos. 2009/0247943 entitled “Robotic Catheter Device Cartridge” and 2009/0247944 entitled “Robotic Catheter Rotatable Device Cartridge,” the entire disclosures of which are incorporated herein by reference.

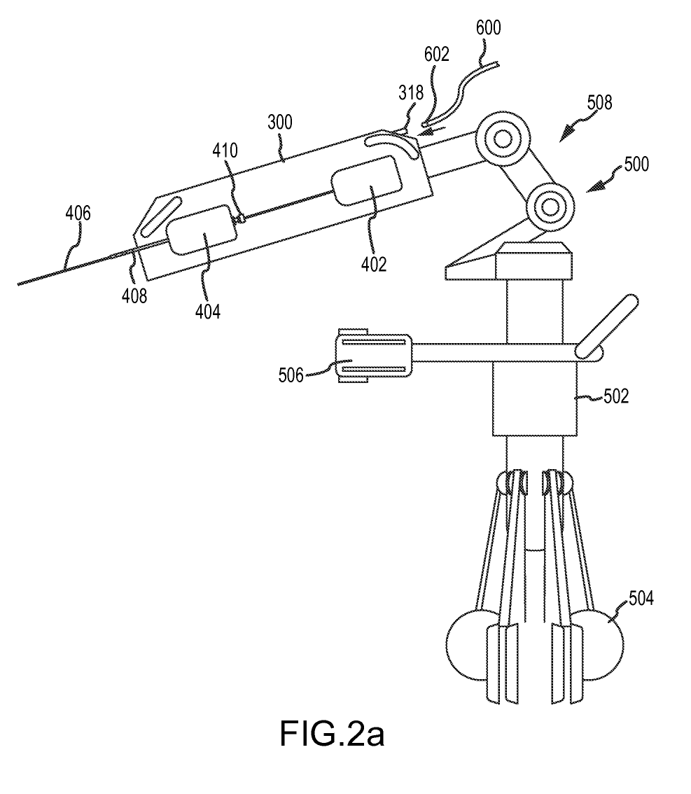

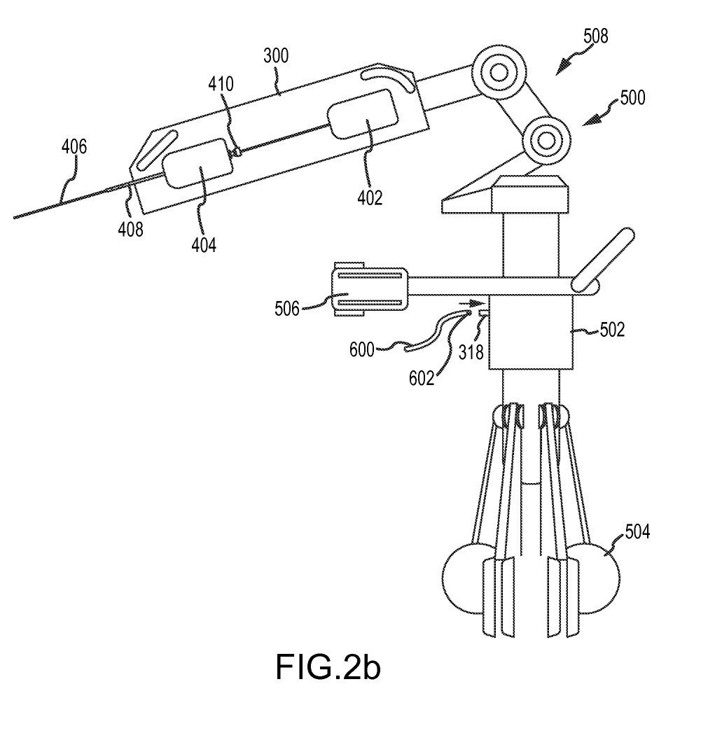

FIGS. 2a and 2b are side views of exemplary manipulator support structures 500 (see U.S. Patent Publication No. 2009/0247944, the entire disclosure of which was incorporated by reference above) for supporting the manipulator assembly 300. The structure 500 can generally include a support frame 502 including retractable wheels 504 and attachment assembly 506 for attachment to an operating bed (not shown). A plurality of support linkages 508 can be provided for accurately positioning one or more manipulator assemblies, such as the manipulator assembly 300.

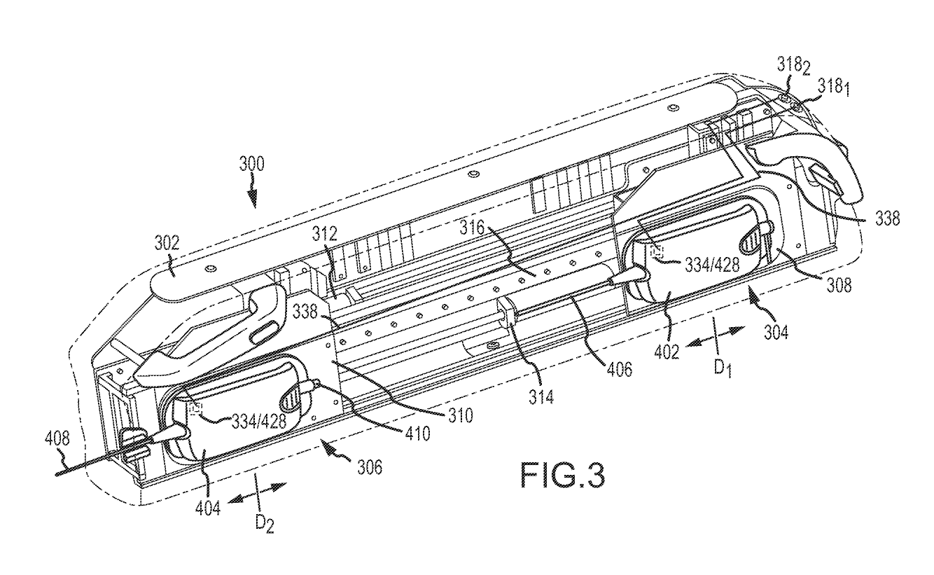

With reference to FIGS. 2a-3, the assembly 300 includes a housing 302 and is configured to serve, for example, as the interface for the mechanical control of the movements or actions of one or more device cartridges, such as catheter and sheath cartridges 402, 404 described below. Each device cartridge is configured to receive and retain a respective proximal end of an associated medical device (e.g., catheter or sheath). The assembly 300 also includes one or more plurality of manipulation bases onto which the device cartridges are mounted or attached. After mounting, the manipulator assembly 302, through the manipulation bases, is capable of manipulating the attached catheter and/or sheath.

FIG. 3 is an isometric view of the manipulator assembly 300 with portions of the housing 302 thereof omitted for clarity. The manipulator assembly 300 includes, in part, a catheter manipulator mechanism 304, a sheath manipulator mechanism 306, a catheter manipulation base 308, a sheath manipulation base 310, a first (catheter) drive mechanism 312, a second (sheath) drive mechanism 314, and a track 316, all disposed within the housing 302.

With reference to FIGS. 2a, 3, and 4, in an exemplary embodiment, the manipulator assembly 300 further includes one or more electrical ports 318 disposed in the housing 302. In the illustrated embodiment, the manipulator assembly 300 includes two electrical ports 318 (318₁ and 318₂), each of which has a first end that is configured to be mated with a corresponding and complementary connector of a cable or wire. As will be described below, the electrical ports 318 allow for the connection of certain components of the RCGS 10 associated with the manipulator assembly 300 to be electrically connected to other components of, or used with, the RCGS 10, such as, for example, modeling or mapping systems, generators, stimulators, and the like. Accordingly, the electrical ports 318 are configured to be mated or coupled with complementary electrical connectors of cables or wires that are connected to, or configured to be connected to, other components.

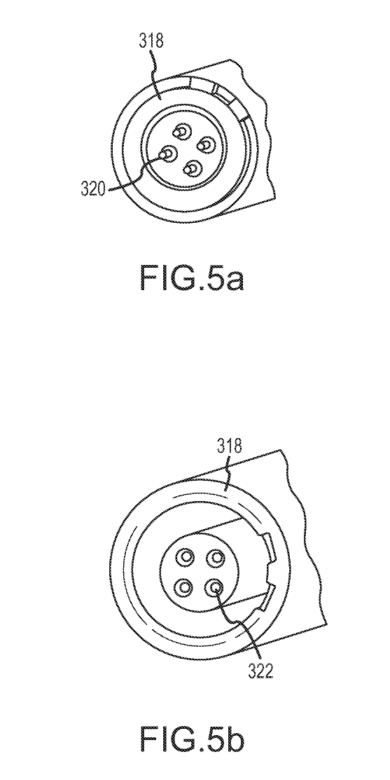

The electrical ports 318 may take any number of forms known in the art, such as, for example and without limitation, those illustrated in FIGS. 5a and 5b. FIG. 5a depicts an exemplary port 318 comprising a male plug connector having a plurality of pin contacts 320 (e.g., a first end of the port 318 comprises the male plug connector). In such an embodiment, the port 318 is configured to be mated with a complementary female receptacle connector having a plurality of socket contacts configured to receive the pin contacts 320 of the male plug connector. Alternatively, FIG. 5b depicts another exemplary port 318 comprising a female receptacle connector having a plurality of socket contacts 322 configured to receive a corresponding number of pin contacts from a complementary male plug connector of a cable. In an exemplary embodiment, the port(s) 318 of the manipulator assembly 300 is/are keyed so as to prevent the mating of electrical connectors other than correctly-oriented matching connectors.

While male plug and female receptacle connectors are described herein with specificity, it will be appreciated by those of ordinary skill in the art that the port(s) 318 may take the form of any connectors and any number of connections as is known in the art in addition to those described with particularity herein, and such alternative connectors remain within the spirit and scope of the present disclosure.

While the ports 318 have a first end that is configured for connection with a cable or wire that is disposed exterior to the housing 302, each port 318 further includes a second end that is disposed within the housing 302. As will be described in greater detail below, the second end of each port 318 is electrically connected to a respective electrical interface or element disposed in or on the manipulator assembly 300, the device cartridge 400, the catheter 406, the sheath 408, or one or more components thereof.

While the description has thus far been with respect to an embodiment wherein the manipulator assembly 300 includes one or more ports 318 in the housing 302 thereof, in another exemplary embodiment, such as, for example, that illustrated in FIG. 2b, the support structure 500 may also, or alternatively, include one or more ports 318 therein. More particularly, one or more components (e.g., support frame 502, attachment assembly 506, and/or support linkages 508, for example) of the support structure 500 may include one or more ports 318 therein. It will be appreciated that except as otherwise noted herein, the description of the ports 318 set forth above and below applies with equal weight to such an embodiment, and therefore, the description will not be repeated but rather is incorporated here by reference.

As further shown in FIG. 3, and as will be described in greater detail below, the manipulator assembly 300 further includes a catheter cartridge 402 and a sheath cartridge 404, with a catheter 406 coupled to the catheter cartridge 402 and a sheath 408 coupled to the sheath cartridge 404.

With continued reference to FIG. 3, the catheter and sheath manipulator mechanisms 304, 306 are configured to manipulate the several different movements of the catheter 406 and the sheath 408, respectively. First, each mechanism 304, 306 is respectively configured to impart translation movement to the catheter 406 and the sheath 408. Translation movement here refers to the independent advancement and retraction (withdrawal) as shown generally in the directions designated D1 and D2 in FIG. 3. Second, each mechanism 304, 306 is also configured to effect deflection of the distal end of either or both of the catheter and sheath 406, 408. Third, each mechanism 304, 306 can be operative to effect a so-called virtual (omni-directional) rotation of the distal end portions of the catheter 406 and the sheath 408. Virtual rotation, for example, can be made through the use of independent four-wire steering control for each device (e.g., eight total steering wires, comprising four sheath control wires and four catheter control wires). The distal end movement is referred to as “virtual” rotation because the outer surface of the medical device does not in fact rotate in the conventional sense (i.e., about a longitudinal axis) but rather achieves the same movements as conventional uni-planar deflection coupled with axial rotation. In addition to the present description of virtual rotation, further details can be found in PCT/US2009/038597 entitled “Robotic Catheter System with Dynamic Response,” published as WO 2009/120982 and incorporated herein by reference above.

Each manipulator mechanism 304, 306 further includes a respective manipulation base 308, 310 onto which are received the catheter and sheath cartridges 402, 404 (i.e., the catheter and sheath cartridges 402, 404 are configured to be removably attached to the manipulation bases 308, 310, respectively). Each interlocking base 308, 310 can be capable of travel in the longitudinal direction of the catheter/sheath (i.e., D1, D2 respectively) along a track 316. In an embodiment, D1 and D2 can each represent a translation of approximately eight (8) linear inches. Each interlocking base 308, 310 can be translated by respective high precision drive mechanisms 312, 314. Such drive mechanisms can include, for example and without limitation, an electric motor driven lead screw or ball screw.

In the embodiment illustrated in FIG. 3, the manipulator mechanisms 304, 306 are aligned with each other such that catheter 406 can pass through sheath 408 in a coaxial arrangement. Thus, sheath 408 can include a water-tight proximal sheath opening 410 sized to receive the catheter 406 and to let the catheter 406 pass therethrough. Overall, the manipulator mechanisms 304, 306 are configured to allow not only coordinated movement but also relative movement between catheter and sheath cartridges 402, 404 (and thus relative movement between catheter and sheath).

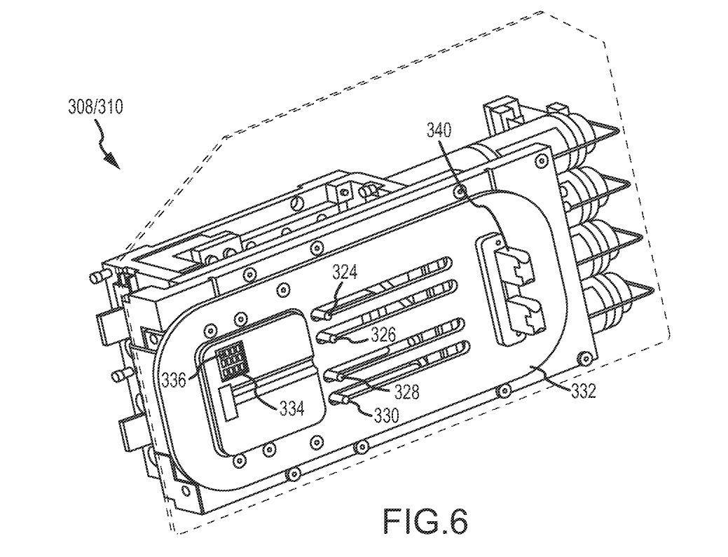

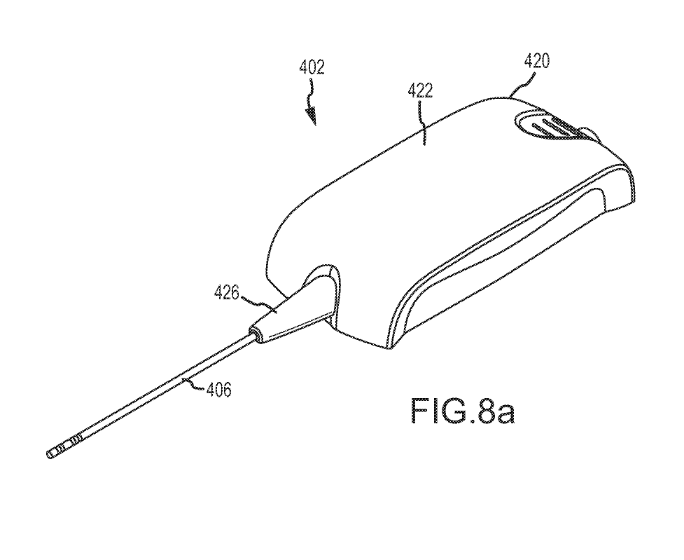

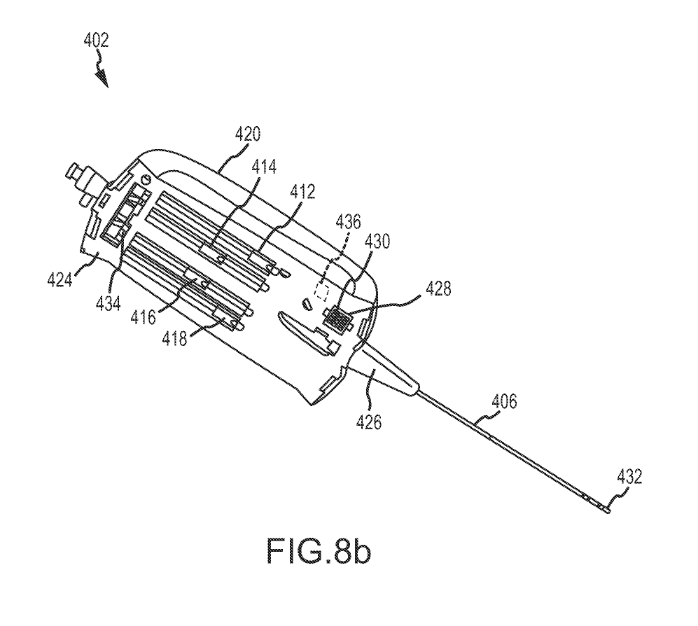

FIG. 6 is an isometric, enlarged view showing the manipulation base 308 (and base 310) in greater detail. The manipulator assembly 300 includes a manipulation base for each cartridge of the RCGS 10. Accordingly, in the embodiment described herein that includes both a catheter and a sheath, the manipulator assembly 300 includes two manipulation bases—the manipulation base 308 for and corresponding to the catheter cartridge 402, and the manipulation base 310 for and corresponding to the sheath cartridge 404. Each base 308, 310 can include a plurality of fingers 324, 326, 328, 330 (e.g., one per steering wire) that extend or protrude upwardly to contact and interact with steering wire slider blocks of the cartridges (i.e., such as slider blocks 412, 414, 416, 418 of the cartridges shown in FIG. 8b) to independently tension select steering wires of the respective medical device. Each finger can be configured to be independently actuated (i.e., moved back and forth within the oval slots depicted in FIG. 6) by a respective precision drive mechanism, such as a motor driven ball screw. A plate 332 of the manipulation base provides a surface onto which a cartridge is seated.

In an exemplary embodiment, each of the manipulation bases 308, 310 further comprises an electrical interface 334. Each electrical interface 334 is configured to electrically contact a complementary electrical interface of either the catheter cartridge 402 (in the case of the electrical interface 334 of the manipulation base 308), or the sheath cartridge 404 (in the case of the electrical interface 334 of the manipulation base 310) when the catheter and sheath cartridges 402, 404 are attached to the manipulation bases 308, 310. Each of the electrical interfaces 334 comprises a plurality of electrical contacts 336. The number of electrical contacts 336 that each electrical interface 334 has may vary depending on the application and/or the particular catheter or sheath associated with the cartridges 402, 404. In an exemplary embodiment, each electrical interface 334 comprises a contact pad disposed in or on the respective plates 332 of the manipulation bases 308, 310. In such an embodiment, the contact pads are sized so as to have enough surface area to make good electrical contact with the corresponding electrical interfaces of the cartridges 402, 404, and are precisely placed or located on the respective plates 332 to allow for mechanical alignment and reliable electrical contact.

It will be appreciated that while the description of the electrical interfaces 334 has thus far been with respect to the electrical interfaces comprising contact pads, the present disclosure is not meant to be so limited. Rather, in other exemplary embodiments, the electrical interfaces 334 may take the form of any number of electrical interfaces known in the art other than contact pads, such as, for example and without limitation, pin and socket connectors, spring contacts, optical interfaces, or radio-frequency identification (RFID) interfaces. Accordingly, embodiments wherein the electrical interfaces 334 are other than those described with specificity herein remain within the spirit and scope of the present disclosure.

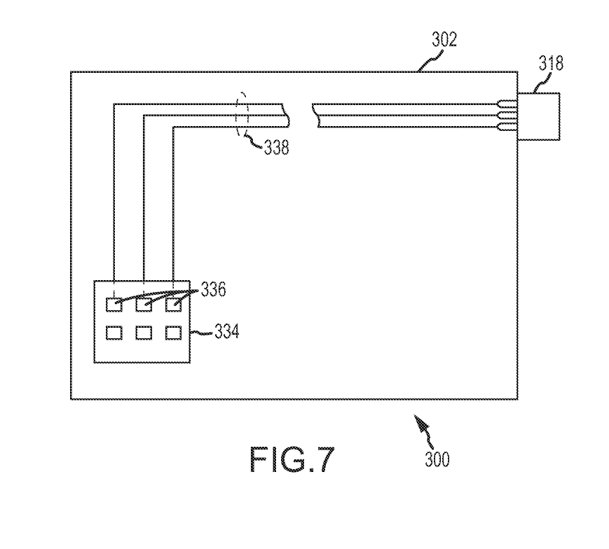

With reference to FIGS. 3 and 7, each of the electrical interfaces 334 is electrically connected to a corresponding electrical port 318, and to an end (second end) thereof that is disposed within the housing 302, in particular. It will be appreciated that in an embodiment wherein the support structure 500 includes one or more ports 318, the second end of the port 318 is disposed within the interior of one of the components of the support structure 500. In any event, in an exemplary embodiment, one port 318 (e.g., 318₁) is electrically connected to the electrical interface 334 disposed in or on the manipulation base 308 and another port 318 (e.g., 318₂) is electrically connected to the electrical interface 334 disposed in or on the manipulation base 310. More particularly, some or all of the electrical contacts 336 of the electrical interfaces 334 are electrically connected to the ports 318, and to respective electrical contacts of the ports 318, in particular (e.g., either pin contacts or socket contacts of the ports in an embodiment wherein the ports take the form of male plug or female receptacle connectors). In an exemplary embodiment, the electrical contacts 336 of the electrical interfaces 334 are electrically connected to respective contacts of the ports 318 by a single multi-conductor cable, wherein each conductor of the cable connects a single electrical contact 336 of an electrical interface 334 with a single electrical contact of a corresponding port 318. Alternatively, the electrical contacts 336 of the electrical interfaces 334 may be electrically connected to respective contacts of corresponding ports 318 by individual electrical wires, whereby each individual wire connects a single electrical contact 336 of an electrical interface 334 with a single electrical contact of a corresponding port 318. In either instance, the cable or wires, collectively designated as reference numeral 338 in FIGS. 3 and 7, are routed between the electrical interfaces 334 and the ports 318 (i.e., the wires 338 are routed within and through the housing 302 of the manipulator assembly 300, and/or the interior of one or more components of the support structure 500, in an embodiment wherein the support structure includes one or more ports, to the ports 318).

In an exemplary embodiment, one or more of the electrical contacts 336 of the electrical interface(s) 334 are configured to be used as a contact sensor so as to provide an indication to the RCGS 10 (either circuitry of the manipulator assembly 300 disposed within the housing 302, or other components of the RCGS 10 (e.g., the electronic control system 200) through the ports 318) that a cartridge has been attached to that corresponding manipulation base 308, 310. Accordingly, in such an embodiment, when a cartridge is attached to a manipulation base, the electrical contacts 336 of the electrical interface 334 makes electrical contact with corresponding electrical contacts of the electrical interface of the cartridge. A signal indicative of the attachment is then generated and sent to the appropriate component of the RCGS 10. It will be appreciated that in other exemplary embodiments, other types of sensors may be used to sense the attachment of a cartridge to a manipulation base (e.g., magnetic sensors and optical sensors). In such embodiments, the sensor(s) may be electrically connected to a port 318 and/or circuitry of the manipulator assembly 300 that is disposed within the housing 302 to provide an indication to either the circuitry of the manipulator assembly 300 and/or other components of the RCGS 10 (e.g., the electronic control system 200) that a cartridge has been attached to that corresponding manipulation base.

It will be appreciated by those of ordinary skill in the art that in practice, the manipulation bases 308, 310 will include structure or components in addition to that or those described herein that are not pertinent for the purposes of this disclosure. For example, the manipulation bases 308, 310 may further comprise electric motors and/or other drive mechanisms to allow for the manipulation of the medical devices that are associated with the cartridge that is attached thereto. Additionally, the manipulation bases 308, 310 may include structure to allow the manipulation bases 308, 310 to move along the track 316. Such additional components may include, for example, those described in the references incorporated by reference above, and/or in U.S. patent application Ser. No. 12/982,964 filed on Dec. 31, 2010, the entire disclosure of which is incorporated herein by reference.

FIG. 8a is an isometric view showing a catheter cartridge 402. It should be understood that the description of the catheter cartridge 402, except as otherwise stated, applies equally to the sheath cartridge 404. The catheter and sheath cartridges 402, 404 each include a housing 420 having a first or top side 422, and a second or bottom side 424 (best shown in FIG. 8b). The proximal ends of the catheter 406 and the sheath 408 are substantially connected or affixed to, and retained within, the respective housings 420 of the cartridges 402, 404 (e.g., in the neck portions 426 of the housings 420). Thus, advancement or retraction of the cartridge 402 correspondingly advances or retracts the catheter 406, and the advancement or retraction of the cartridge 404 correspondingly advances or retracts the sheath 408.

FIG. 8b is an isometric view showing, in greater detail, the bottom side 424 of the catheter cartridge 402. The cartridge 402 can include slider blocks (e.g., slider blocks 412, 414, 416, 418), each rigidly and independently coupled to a respective one of a plurality of steering wires of the catheter 406 in a manner that permits independent tensioning of each steering wire. Likewise, the cartridge 404 for the sheath 408 also includes slider blocks for coupling to a plurality (e.g., four) steering wires of the sheath 408. The device cartridges 402, 404 can be provided as disposable items that are capable of being easily positioned (e.g., removably snapped or attached) into place onto the corresponding manipulation bases 308, 310.

In an exemplary embodiment, each of the catheter and sheath cartridges 402, 404 further comprises an electrical interface 428 disposed in or on the bottom sides 424 of the respective housings 420 thereof. The electrical interfaces 428 are configured to be complementary to, and to electrically contact, respective electrical interfaces 334 of the manipulation bases 308, 310 described above when the cartridges 402, 404 are attached to the manipulation bases 308, 310. The electrical interfaces 428 of the cartridges 402, 404 each comprise a plurality of electrical contacts 430. The number of electrical contacts 430 for each electrical interface 428 may vary depending on the application and/or the particular catheter or sheath associated with the cartridges 402, 404. In an exemplary embodiment, each electrical interface 428 comprises a contact pad disposed in or on the bottom side 424 of the housing 420 of the corresponding cartridge 402, 404. In such an embodiment, the contact pads are each sized so as to have enough surface area to make good electrical contact with the respective electrical interfaces 334 of the manipulation bases 308, 310, and are precisely placed or located on the bottom sides 424 of the housings 420 to allow for mechanical alignment and reliable electrical contact.

As with the electrical interfaces 334 described above, it will be appreciated that while the description of the electrical interfaces 428 has thus far been with respect to the electrical interfaces comprising contact pads, the present disclosure is not meant to be so limited. Rather, in other exemplary embodiments, the electrical interfaces 428 may take the form of any number of electrical interfaces known in the art other than contact pads, such as, for example and without limitation, pin and socket connectors, or spring contacts. Accordingly, embodiments wherein the electrical interfaces 428 are other than those described with specificity herein remain within the spirit and scope of the present disclosure.

With continued reference to FIG. 8b, the catheter 406 associated with the cartridge 402 includes one or more sensors 432 disposed at or near the distal end thereof. Similarly, the sheath 408 associated with the cartridge 404 may also include one or more sensors 432 disposed at or near the distal end thereof. Whether part of the catheter 406 and/or the sheath 408, the sensors 432 may be provided for a variety of diagnostic and therapeutic purposes including, for example, EP studies, catheter identification and location, pacing, cardiac mapping, ablation, and the like. Additionally, the sensors 432 may take any number of forms, such as, for example, tip electrodes, ring electrodes, button electrodes, coil electrodes, brush electrodes, flexible polymer electrodes, and spot electrodes, to name a few. It will be appreciated that the number, shape, orientation, and purpose of the sensor(s) 432 may vary.

Each sensor 432 of the catheter 406 and/or sheath 408 includes a corresponding lead wire. The lead wires are configured to electrically connect the sensor(s) 432 associated with the catheter 406 and/or sheath 408 with corresponding electrical contacts 430 of the electrical interfaces 428 of the cartridges 402, 404, respectively. Accordingly, a lead wire corresponding to a sensor 432 of the catheter 406 is electrically connected between that sensor 432 and an electrical contact 430 of the electrical interface 428 of the cartridge 402, while a lead wire corresponding to a sensor 432 of the sheath 408 is electrically connected between that sensor 432 and an electrical contact 430 of the electrical interface 428 of the cartridge 404. Thus, the lead wires of the sensor(s) 432 extend through one or more lumens of the catheter 406 or sheath 408 from a corresponding sensor 432, into the housing 420 of the cartridge 402 or cartridge 404, and to the electrical interface 428.

Referring to FIGS. 6 and 8b, the catheter and sheath cartridges 402, 404 are configured to be attached or secured onto respective manipulation bases 308, 310. To couple the cartridge 402 (and 404) with the base 308 (and 310), in an exemplary embodiment, one or more locking pins (e.g., 340 in FIG. 6) on the manipulation base can engage one or more mating recesses or sockets 434 in the cartridge (see FIG. 8b). In an embodiment, such recesses 434 can include an interference lock such as a spring detent or other locking means. In an embodiment, such other locking means can include a physical interference that can require affirmative/positive action by the user to release the cartridge from the manipulation base. Such action can include or require actuation of a release lever. Additionally, the cartridge or the manipulation base can include one or more locator pins (not shown) configured to passively fit into mating holes (not shown) on the base or the cartridge, respectively.

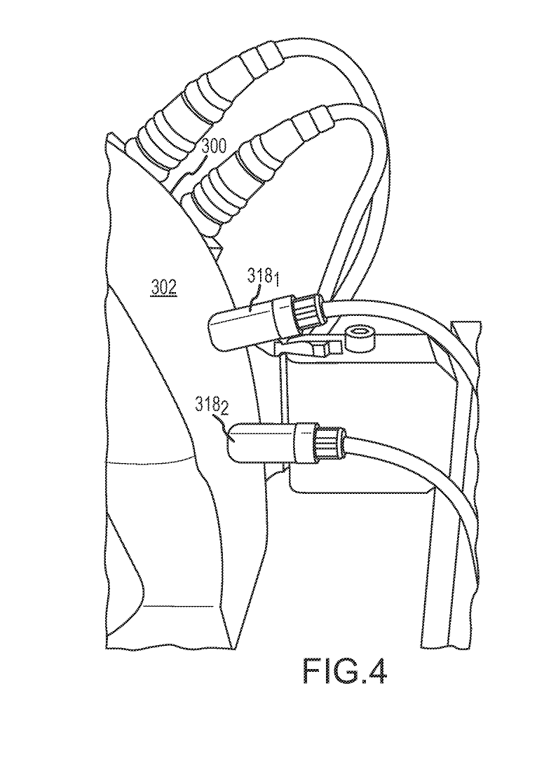

When the catheter and sheath cartridges 402, 404 are attached or secured onto respective manipulation bases 308, 310, and as briefly described above, the electrical interfaces 428 of the cartridges 402, 404 are each aligned and in electrical contact with respective electrical interfaces 334 of the manipulation bases 308, 310. Accordingly, by virtue of the respective electrical connections between the electrical interfaces 428 and the sensor(s) 432, and the electrical interfaces 334 and the ports 318, as well as the electrical connections between the electrical interfaces 334, 428 themselves, the sensor(s) 432 can be electrically connected to the ports 318. Thus, when cables or wires, such as, for example the cable(s) (or wire(s)) 600 illustrated in FIGS. 2a, 2b, and 4, are mated with the ports 318₁, 318₂, electrical signals sensed or produced by the sensor(s) 432 can be communicated to the components of, or used with, the RCGS 10 to which those cables or wires 600 are connected, such as, for example, the electronic control system 200, and may, therefore, be used in the operation of the RCGS 10. Accordingly, in such an embodiment, the cables 600, through the respective electrical connectors 602 thereof, are configured to electrically connect the port(s) 318 with one or more components of, or used with, the RCGS 10, such as, for example, the electronic control unit 200, the visualization, navigation, and mapping system 14, ablation generators, stimulators, ultrasound systems, one or more break out boxes configured to split and direct the signals in the appropriate direction, and the like.

For example, one important electrical parameter the RCGS 10 evaluates during operation is the tissue impedance. The tissue impedance is used by the RCGS 10, and the electronic control system 200 and/or the visualization, navigation, and mapping system 14 thereof, in particular, to prevent tissue perforation. One or more of the sensors 432 disposed in or on the catheter 406 and/or sheath 408 can be used to acquire data relating to this impedance. Electrical signals produced by there sensor(s) 432, and the data represented thereby, can then be communicated from the sensor(s) 432 through the electrical interfaces 334, 428 of the corresponding cartridge and manipulation base, through the corresponding port 318, and onto the appropriate component of the RCGS 10 that uses this data in the control and guidance of the catheter 406 and/or sheath 408 through the electrical cables 600. More particularly, the signals may be communicated from the sensor(s) 432, through the interfaces 334, 428, through the wire(s) 338 connecting the interface 334 with the port 318, through the port 318, and then onto the RCGS component through the cable(s) 600 connecting the port 318 with the RCGS component. Accordingly, the electrical interfaces of the cartridges and the manipulation bases, when in electrical contact with each other, provide an electrical connection between the sensors(s) disposed in or on the catheter and/or sheath and other components of, or used with, the RCGS 10, and therefore, provides a means by which, for example, data acquired by the sensor(s) 432 can be quickly and efficiently communicated to other components.

In an exemplary embodiment, one or both of the cartridges 402, 404 may further include a memory or storage device 436, such as, for example and without limitation, an electrically erasable programmable read-only memory (EEPROM), disposed within the housing 420. The memory 436 may contain, for example, identifying information relating to the device associated with the cartridge. The information may comprise for example, the make, model, serial number, physical dimensions, special features, and/or calibration data related to the catheter or sheath. One exemplary purpose of providing this information relates to the instance wherein the use of a cartridge and associated medical device is restricted to a single use. Accordingly, the information contained in the memory 436 may be provided to allow the RCGS 10 to determine, for example, whether the particular catheter or sheath cartridge 402, 404 associated with the memory 436 has been previously used, and if so, to provide an indication to the user to remove that particular cartridge from the manipulator assembly 300.

One or both of the catheter and sheath cartridges 402, 404 may include a corresponding memory 436. In any event, in an exemplary embodiment, each memory 436 is electrically connected to one or more electrical contacts 430 of the electrical interface 428 of the respective cartridges 402, 404. Further, an equal number of electrical contacts 336 of the complementary electrical interface 334 of the corresponding manipulation base(s) 308, 310 will correspond to, and be configured to electrically contact, the electrical contacts 430 of the electrical interface 428 associated with the memory 436.

As was described above, when the cartridge(s) 402, 404 are attached or secured onto respective manipulation bases 308, 310, the electrical interfaces 428 of the cartridges 402, 404 are each aligned and in electrical contact with respective electrical interfaces 334 of the manipulation bases 308, 310. Accordingly, by virtue of the electrical connections between the electrical interfaces 428 and the memory (or memories) 436, and the electrical interfaces 334 and the ports 318, as well as the electrical connections between the electrical interfaces 334, 428 themselves, the memory (or memories) 436 may also be electrically connected to the port(s) 318. Thus, when cables or wires, such as cables/wires 600, are mated with the appropriate port(s) 318, the data stored on the memory (or memories) 436 can be communicated to the components of the RCGS 10 to which those cables or wires 600 are connected, such as, for example, the electronic control system 200, and can be used in the operation of the RCGS 10. In addition, or alternatively, the electrical contacts 430 of the electrical interface 428 to which a memory 436 is connected may be connected to other circuitry disposed within the housing 302 of the manipulator assembly 300, and may be used in much the same manner as that described above.

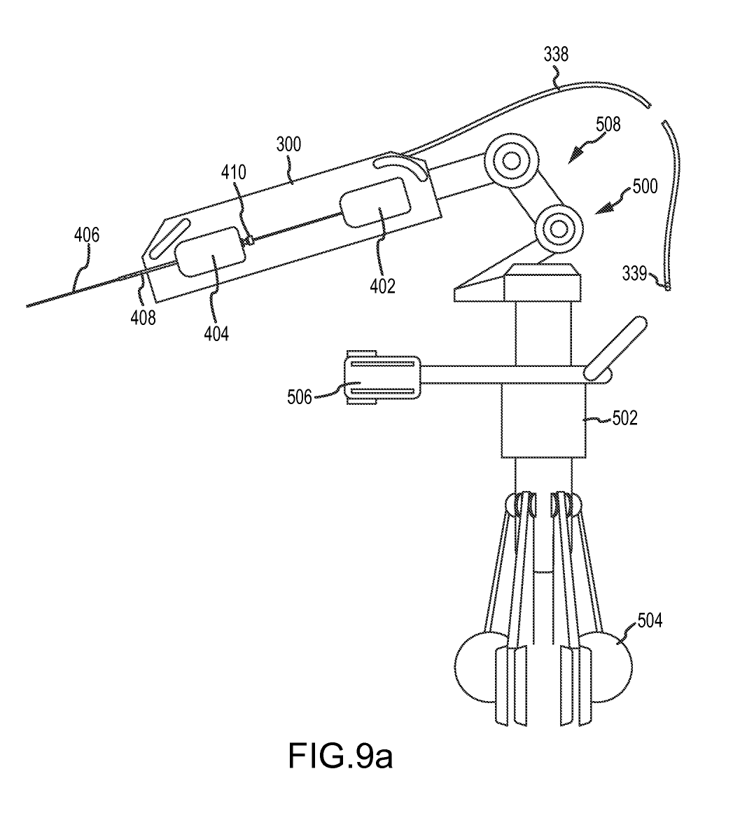

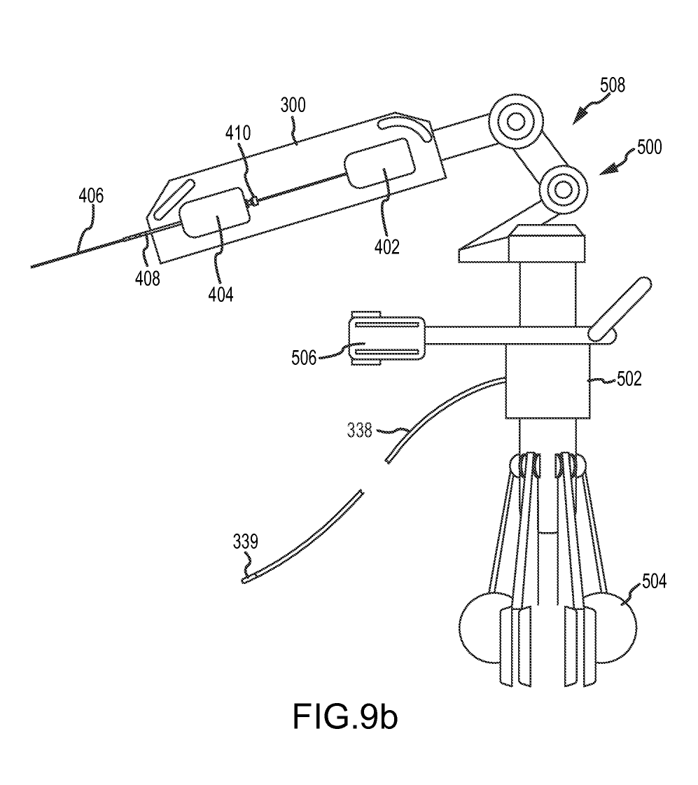

While the description above has thus far been with respect to one or more cables 338 connecting one or more electrical interfaces 334 with one or more ports 318, in another exemplary embodiment, the cable(s) 338 is/are not electrically coupled to port(s) 318, but rather directly connect the electrical interface(s) 334 with one or more components of, or used with, the RCGS 10. More particularly, and with reference to FIGS. 9a and 9b, in an embodiment, one or more cables 338 each have a first end that is coupled to an electrical interfaces 334 (e.g., each cable is electrically coupled to either the interface 334 of the manipulation base 308, or the interface 334 of the manipulation base 310), and a second end that has an electrical connector 339 electrically coupled thereto, which may comprise any number of types of electrical connectors known in the art, including, for example and without limitation, those described elsewhere herein.

In such an embodiment, and as illustrated in FIG. 9a, one or more cables 338 may extend from the housing 302 of the manipulator assembly 300 and each is configured to be directly coupled, via the electrical connector 339 thereof, with one or more components of, or used with, the RCGS 10, such as, for example, the electronic control unit 200, the visualization, navigation, and mapping system 14, ablation generators, stimulators, ultrasound systems, one or more break out boxes configured to split and direct the signals in the appropriate direction, and the like. In other words, the electrical connector 339 of the cable 338 is configured to be mated with a complementary electrical connector of the component. Alternatively, the cable(s) 338 may be electrically coupled to another cable or wire, that in turn, extends from the housing 302 to the component.

In another exemplary embodiment, such as that illustrated in FIG. 9b, one or more cables 338 may extend through the housing 302 of the manipulator assembly 300 and/or through the support structure 500, and one or more components thereof, in particular (e.g., support frame 502, attachment assembly 506, support linkages 508, for example). In such an embodiment, one or more cables 338 may extend from the support structure 500 and each is configured to be directly coupled, via the electrical connector 339 thereof, with one or more components of, or used with, the RCGS 10, such as, for example, the electronic control unit 200, the visualization, navigation, and mapping system 14, ablation generators, stimulators, ultrasound systems, one or more break out boxes configured to split and direct the signals in the appropriate direction, and the like. In other words, the electrical connector 339 of the cable 338 is configured to be mated with a complementary electrical connector of the component. Alternatively, the cable(s) 338 may be electrically coupled to another cable, that in turn, extends from the support structure 500 to the component.

In operation, a user first manually positions catheter 406 and sheath 408 (with catheter 406 inserted in sheath 408) within the vasculature of a patient. Once the medical devices are roughly positioned in relation to the heart or other anatomical site of interest, the user can then engage or connect (e.g., “snap-in”) the catheter and sheath cartridges into place on respective bases 308, 310. When a cartridge is interconnected with a base, the fingers thereof fit into the recesses formed in the slider blocks. For example, with respect to the catheter cartridge 402 and the corresponding manipulation base 308, each of the plurality of fingers 316, 318, 320 or 322 fit into corresponding recesses or slots formed in the slider blocks 412, 414, 416, 418 (best shown in FIG. 8b). Each finger can be designed to be actuated to respectively move each slider block, thereby placing the respective steering wire in tension (i.e., a “pull” wire). Translation, distal end bending and virtual rotation can be accomplished through the use of the RCGS 10. Further, by virtue of the electrical connections between the electrical interfaces of a cartridge and a corresponding manipulation base, information and/or data from the cartridge, the medical device associated therewith, the manipulation base, and/or the manipulator assembly 300 can be efficiently and quickly communicated to the balance of the RCGS 10, thereby improving the performance of the RCGS 10.

Although only certain embodiments have been described above with a certain degree of particularity, those skilled in the art could make numerous alterations to the disclosed embodiments without departing from the scope of this disclosure. Joinder references (e.g., attached, coupled, connected, and the like) are to be construed broadly and may include intermediate members between a connection of elements and relative movement between elements. As such, joinder references do not necessarily infer that two elements are directly connected/coupled and in fixed relation to each other. Additionally, all directional references (e.g., top and bottom) are only used for identification purposes to aid the reader's understanding of the present invention, and do not create limitations, particularly as to the position, orientation, or use of the invention. Further, the terms electrically connected and in communication are meant to be construed broadly to encompass both wired and wireless connections and communications. It is intended that all matter contained in the above description or shown in the accompanying drawings shall be interpreted as illustrative only and not limiting. Changes in detail or structure may be made without departing from the invention as defined in the appended claims.

What is claimed is:

1. An apparatus for a robotically controlled medical device guidance system, comprising:
a manipulator assembly having a housing and a manipulation base disposed within said housing, wherein said manipulation base includes an electrical interface;
an electrical port having a first end and a second end, wherein said first end is configured for mating with a complementary electrical connector of a cable, and said second end is electrically connected to said electrical interface of said manipulation base; and
a medical device cartridge configured to be removably attached to said manipulation base, said medical device cartridge having:
an electrical interface configured to electrically contact said electrical interface of said manipulation base when said medical device cartridge is attached to said manipulation base; and
a medical device associated therewith, said medical device including a sensor and a corresponding lead wire, said lead wire being electrically connected to and between said sensor and said electrical interface of said medical device cartridge.

2. The apparatus of claim 1, wherein said medical device cartridge is removably attached to said manipulation base, and said electrical interface of said medical device cartridge is in electrical contact with said electrical interface of said manipulation base, and further wherein said sensor of said medical device is electrically connected to said electrical port through the electrical connection between said respective electrical interfaces of said medical device cartridge and said manipulation base.

3. The apparatus of claim 1, wherein said electrical port is a first electrical port, said manipulation base is a first manipulation base, and said medical device cartridge is a first medical device cartridge, and further wherein:
said manipulator assembly further comprises a second manipulation base disposed within said housing, said second manipulation base including an electrical interface; and
said apparatus further comprises:
a second electrical port having a first end and a second end, wherein said first end is configured for mating with a complementary electrical connector of a cable, and said second end is electrically connected to said electrical interface of said second manipulation base; and
a second medical device cartridge configured to be removably coupled with said second manipulation base, said second medical device cartridge having:
an electrical interface configured to electrically contact said electrical interface of said second manipulation base when said second medical device cartridge is attached to said second manipulation base; and
a medical device associated therewith, said medical device including a sensor and a corresponding lead wire, said lead wire being electrically connected to and between said sensor and said electrical interface of said second medical device cartridge.

4. The apparatus of claim 3, wherein said first medical device cartridge is removably attached to said first manipulation base, and said second medical device cartridge is removably attached to said second manipulation base, and further wherein:
said electrical interface of said first medical device cartridge is in electrical contact with said electrical interface of said first manipulation base, and further wherein said sensor of said medical device of said first medical device cartridge is electrically connected to said second end of said first electrical port through the electrical connection between said electrical interfaces of said first medical device cartridge and said first manipulation base; and
said electrical interface of said second medical device cartridge is in electrical contact with said electrical interface of said second manipulation base, and further wherein said sensor of said medical device of said second medical device cartridge is electrically connected to said second end of said second electrical port through the electrical connection between said electrical interfaces of said second medical device cartridge and said second manipulation base.

5. The apparatus of claim 3, wherein said medical device of said first medical device cartridge is a sheath and said medical device of said second medical device cartridge is a catheter.

6. The apparatus of claim 1, wherein said medical device cartridge further comprises a memory containing identifying data relating to said medical device, said memory being electrically connected to said electrical interface of said medical device cartridge.

7. The apparatus of claim 6, wherein said memory is an electrically erasable programmable read-only memory (EEPROM).

8. The apparatus of claim 1, wherein said manipulation base further comprises a sensor configured to detect the attachment of said medical device cartridge.

9. The apparatus of claim 1, wherein said electrical interfaces of said medical device cartridge and said manipulation base each comprise a contact pad having a plurality of complementary electrical contacts disposed thereon.

10. The apparatus of claim 1, wherein one of said electrical interfaces of said medical device cartridge and said manipulation base comprises at least one pin, and the other of said electrical interfaces comprises at least one complementary socket configured to receive said at least one pin.

11. The apparatus of claim 1, wherein said port is disposed in said housing of said manipulator assembly.

12. The apparatus of claim 1 further comprising a support structure for supporting said manipulator assembly, and wherein said port is disposed in said support structure.

13. An apparatus for a robotically controlled medical device guidance system, comprising:
a housing;
an electrical port disposed in said housing, said electrical port having a first end and a second end, and wherein said first end is configured for mating with a complementary electrical connector of a cable; and
a manipulation base disposed within said housing, said manipulation base configured to have a medical device cartridge removably attached thereto and including an electrical interface configured to electrically contact a corresponding electrical interface of said medical device cartridge when said medical device cartridge is attached thereto, wherein said electrical interface of said manipulation base is electrically connected to said second end of said electrical port.

14. The apparatus of claim 13, wherein said electrical port is a first electrical port, and said manipulation base is a first manipulation base, said assembly further comprising:
a second electrical port disposed in said housing, said second electrical port having a first end and a second end, and wherein said first end is configured for mating with a complementary electrical connector of a cable; and
a second manipulation base disposed within said housing, said second manipulation base configured to have a medical device cartridge removably attached thereto and including an electrical interface configured to electrically contact a corresponding electrical interface of said medical device cartridge when said medical device cartridge is attached to said second manipulation base, wherein said electrical interface of said second manipulation base is electrically connected to said second end of said second electrical port.

15. The apparatus of claim 13, wherein electrical interface of said manipulation base comprises a contact pad having a plurality of electrical contacts.

16. The apparatus of claim 13, wherein said electrical interface of said manipulation base comprises one of a plurality of pins and a plurality of sockets.

17. A medical device cartridge for a robotically controlled medical device guidance system, comprising:
a housing having a top side and a bottom side, said bottom side configured to permit said medical device cartridge to be removably attached to a manipulator assembly of said robotically controlled medical device guidance system;
an electrical interface disposed on said bottom side of said housing and configured to electrically contact a corresponding electrical interface associated with said manipulator assembly when said medical device cartridge is attached thereto; and
a medical device having a proximal end and a distal end, said proximal end of said medical device being retained within said housing, said medical device further including a sensor and a corresponding lead wire, said lead wire being electrically connected to and between said sensor and said electrical interface.

18. The cartridge of claim 17, further comprising a memory containing identifying data relating to said medical device, said memory being electrically connected to said electrical interface of said medical device cartridge.

19. The cartridge of claim 18, wherein said memory is an electrically erasable programmable read-only memory (EEPROM).

20. The cartridge of claim 17, wherein said medical device comprises a catheter.

21. The cartridge of claim 17, wherein said electrical interface comprises a contact pad having a plurality of electrical contacts.
